(19) United States
(12) Patent Application Publication
(10) Pub. No.: US 2024/0207111 A1
(43) Pub. Date: Jun. 27, 2024

(54) PANTILESS LINER

(71) Applicant: FEME, LLC, Woodland Hills, CA (US)

(72) Inventor: Tara Monique Harris, Woodland Hills, CA (US)

(21) Appl. No.: 18/598,814

(22) Filed: Mar. 7, 2024

Related U.S. Application Data
(63) Continuation-in-part of application No. 17/239,156, filed on Apr. 23, 2021, now abandoned.
(60) Provisional application No. 63/199,479, filed on Dec. 31, 2020.

(51) Int. Cl. A61F 13/56; A61F 13/15; A61F 13/472; A61F 13/513; A61F 13/514; A61F 13/53; A61F 13/84; B01J 20/26; B01J 20/28

(52) U.S. Cl. CPC A61F 13/5605; A61F 13/47209; A61F 13/51456; B01J 20/267; B01J 20/28035; A61F 2013/15365; A61F 2013/51383; A61F 2013/530481; A61F 2013/8497

(57) ABSTRACT

The present disclosure provides for a pantiless liner for a user and a method of positioning the pantiless liner. The pantiless liner can include a moisture-impervious layer, a core, a transfer adhesive layer, a body adhesive layer, and a protective layer. The core can include a super absorbent polymer (SAP) layer and a fabric layer. The body adhesive layer can be disposed on a perimeter of the fabric layer. The body adhesive layer can be configured to contact the user and to adhere to the body. The method can include positioning the pantiless liner lengthwise such that an inner boundary between the fabric layer and the body adhesive layer is positioned adjacent to a clitoral hood of user and such that a rear portion of the pantiless liner is positioned on a portion of a perineum of the user.

### CROSS-REFERENCE TO RELATED APPLICATIONS

This application is a continuation-in-part of U.S. Utility application Ser. No. 17/236,159, filed on Apr. 23, 2021, which claims priority to U.S. Provisional Application No. 63/199,479 filed on Dec. 31, 2020. The entire disclosure of the above application is hereby incorporated herein by reference.

### FIELD

The present technology relates to female hygiene products, more specifically, a panty liner for use without undergarments.

### INTRODUCTION

This section provides background information related to the present disclosure which is not necessarily prior art.

Other panty liners currently on the market only work while wearing panties or thongs. Vaginal discharge is a very common and completely natural phenomenon. Some women use panty liners to protect their underwear from vaginal discharge and maintain a dry, fresh, and clean feeling throughout the day.

A panty liner is a thin, absorbent pad worn in the gusset of underwear to absorb vaginal discharge or light menstrual flow. Panty liners can be thought of as thin versions of pads used to prevent stains and keep underwear clean. Panty liners come in a variety of shapes, portability options, and sizes, from small, compact panty liners to large, protective panty liners created for heavy vaginal discharge. Some styles of panty liners are designed to fit inside thongs. The purpose of panty liners is to absorb everyday vaginal discharge, light spotting, staining at the beginning and ends of periods, and post-intercourse discharge. Tampons, and menstrual cups can be used with panty liners for extra protection. Some people find panty liners to be more flexible and comfortable than pads.

Vaginal discharge is normal and can occur for a variety of reasons throughout the menstrual cycle (keeping the vagina lubricated, ovulation, and arousal, to name a few). Wearing a panty liner can be helpful for keeping underwear dry and free from stains. Having panty liners on hand may be useful during puberty, as spotting and unanticipated periods can be frequent.

Panty liners can be worn before or after periods, but they are not meant for heavy flows. Panty liners can rub against the labia, possibly triggering redness, irritation, and itching. Scented panty liners might contain chemicals that can irritate the sensitive tissues around the vagina. Both synthetic-fiber underwear and regular use of panty liners with an impermeable layer block air circulation to the reproductive organs and don't allow sweat to evaporate.

Accordingly, there is a need for a panty liner that does not require the user to wear undergarments while still providing the freedom to stay clean and comfortable.

### SUMMARY

In concordance with the instant disclosure, a panty liner that does not require the user to wear undergarments while still providing the freedom to stay clean and comfortable, has surprisingly been discovered.

The present technology includes articles of manufacture, systems, and processes that relate to a pantiless liner.

The present disclosure provides for a pantiless liner for a user. The pantiless liner can include a front portion and a rear portion. The pantiless liner can include a moisture-impervious layer, a core, a transfer adhesive layer, a body adhesive layer, and a removable protective layer. The moisture-impervious layer can be a bottom-most layer of the pantiless liner. The core can include a super absorbent polymer (SAP) layer and a fabric layer. The SAP layer can include cross-linked polymers configured to absorb liquid. The fabric layer can be disposed adjacent to the SAP layer and can include a non-woven material. The fabric layer can include a lower surface, an upper surface, and a perimeter. The upper surface can include an exposable portion. The transfer adhesive layer can be disposed between the moisture-impervious layer and the core and can couple a portion of the moisture-impervious layer and the core. The body adhesive layer can be disposed on the upper surface of the fabric layer and can be adjacent to the perimeter of the fabric layer of the core. The body adhesive layer can have an inner boundary and an outer boundary. The outer boundary can be coextensive with the perimeter of the fabric layer and the inner boundary can circumscribe the exposable portion of the upper surface of the fabric layer. The body adhesive layer can abut the upper surface of the fabric layer and can be configured to contact and adhere to the user. The removable protective layer can be disposed adjacent to and selectively covering the exposable portion of the upper surface of the fabric layer. The removable protective layer can be removably adhered to the fabric layer by the body adhesive layer.

The present disclosure further provides a method of using a panty liner without an under. The method can include a step of providing the pantiless liner and removing the protective layer of the pantiless liner. The method can include positioning the front portion of the pantiless liner such that the inner boundary of the body adhesive layer is positioned adjacent a clitoral hood of user. The method can include positioning the rear portion the pantiless liner such that the body adhesive layer of the pantiless liner is positioned on a portion of a perineum of the user. The method can also include pressing the body adhesive layer against the user, whereby the pantiless liner is adhered to the user.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a top plan view of a pantiless liner having a silicone or adhesive strips around the border depicted in an exemplary embodiment of an egg shape;

FIG. 2 is a top plan view of the pantiless liner depicted in an exemplary embodiment of an oval shape;

FIG. 3 is a top plan view of the pantiless liner depicted in an exemplary embodiment with wings;

FIG. 4 is a top plan view of the pantiless liner depicted in an exemplary embodiment of a butterfly;

FIG. 5 is a top plan view of the pantiless liner depicted in an exemplary embodiment of a flower;

FIG. 6 is a top plan view of the pantiless liner depicted in an exemplary embodiment of lips;

FIG. 7 is a top plan view of the pantiless liner depicted in an exemplary embodiment of a leaf;

FIG. 8 is a top perspective view of the pantiless liner according to one embodiment of the present disclosure;

FIG. 9 is a bottom perspective view thereof;

FIG. 10 is a top plan view thereof;

FIG. 11 is a bottom plan view thereof;

FIG. 12 is a front elevational view thereof;

FIG. 13 is a side elevational view thereof;

FIG. 14 is a top perspective exploded view depicting the layers of the pantiless liner according to one embodiment of the present disclosure;

FIG. 15A is a schematic front elevation cross-sectional view of the pantiless liner, according to one embodiment of the present disclosure taken at line 15A of FIG. 10;

FIG. 15B is a schematic side elevation cross-sectional view taken view of the pantiless liner, according to one embodiment of the present disclosure taken at line 15B of FIG. 10;

FIG. 16A is a perspective view of the embodiment of FIGS. 8-13, shown being worn by a user;

FIG. 16B is a cross-sectional view of the embodiment of FIGS. 8-13, shown being worn;

FIG. 16C is an enlarged view of a positioning of the pantiless liner as shown in callout 16C of FIG. 16B; and

FIG. 17 is a flow chart depicting a method for positioning the pantiless liner on a user.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Briefly, and in general terms, the present disclosure provides a new and improved article of clothing comprising a pantiless liner having a silicone or adhesive tape around at least a portion of a border of the pantiless liner to promote secureness where the pantiless liner is positioned to cover the vulva and vaginal area. In certain embodiments, the pantiless liner may include wings that can stick to the inner thigh of a user. The following detailed description is of the best currently contemplated modes of carrying out exemplary embodiments of the invention. The description is not to be taken in a limiting sense but is made merely for the purpose of illustrating the general principles of the present invention.

Broadly, an embodiment of the present invention provides an adhesive panty liner with wings. In one aspect the present disclosure may provide a panty liner that has an adhesive tape to cover the vulva and vaginal area and may include wings that will stick to the inner thigh. The pantiless liner requires no panties or undergarment to use. The present disclosure provides a pantiless liner which allows individuals with external female anatomy to feel secure with normal discharge, secretion and moisture of the vagina that most individuals with external female anatomy have when wearing clothing without panties or undergarments.

Other panty liners currently on the market only work with wearing panties or thongs. The pantiless liner may allow women to be pantiless free with leggings, workout wear and the like while still having the comfort of moisture absorption from the pantiless liner brand. The pantiless liner may also help women who wear tight clothing with the discomfort of the thick inseam on most jeans and pants. With the pantiless liner of the present disclosure, women and young ladies all over the world will feel secure and still maintain a sense of dryness with the pantiless liner.

The pantiless liner of the present disclosure differs from and distinguishes over what currently exists. The pantiless liner may allow the pantiless liner to have silicone strips or adhesive tape that is sensitive to the skin on the upside that will stick to the vulva and inner vaginal wall for secure feeling. The pantiless liner of the present disclosure is an unprecedented improvement over what currently exists. All panty liners on the market currently require wearing panties or an undergarment of some sort. Most women and young ladies currently do not wear panties on a daily basis. Most women only wear panties to absorb the discharge, moisture and secretion through the day.

Also, “No Panties Needed” and pantiless liner brand can also produce maxi pads that can be worn without panties using the same concept of the “No Panties Needed” and pantiless liner brand panty liners. They may be called “No Panties Needed” or pantiless pads, pantiless sanitary Napkins brand for maxi pads.

In an exemplary embodiment, the pantiless liner of the present disclosure may include one or more of the following components or elements. The pantiless liner may be constructed and arranged to include silicone strips or adhesive tape which may be on an upper side that will be capable of adhering to stick to the vulva area and inner thigh around the vaginal area. This positioning of the pantiless liner allows for the wearer to stay dry and feel comfortable. The vaginal opening may be covered with the pantiless liner while the adhesive tape that may stick to the inner thigh around the vaginal opening may endure sweat, moisture, and wetness while removing safely without pain or irritation.

The pantiless liner may include any suitable adhesive which is capable of meeting requirements of the right adhesive tape that will stay secure while there may be sweat, moisture, wetness while maintaining a safe and less painful way if removing. The pantiless liner will also need to test whether the adhesive is reusable if removed to urinate.

The structure may be an improvement over existing panty liners already on the market. To make the pantiless liner adhere to the skin, self-adhesive taping or strips that may securely stick to the inner thigh around the vaginal area are used. The pantiless liner may be made in a regular shape of panty liners and thong design or similar to.

In some embodiments, the pantiless liner may comprise pads that women may wear while on their menstrual cycle. The pantiless liner may further include an adhesive and be of the shape and design that may safely and securely protect the women while they are wearing clothing without panties. In an exemplary embodiment the adhesive may be capable of securely remaining in place if removed for urination and may be reliable for use again.

Options may include small, medium or large size panty liners. The pantiless liner may be of any suitable length. In one embodiment, to securely stay in place, the pantiless liner may require 2 sets of adhesive applied to both sides in the front, middle and toward the back near the rectum.

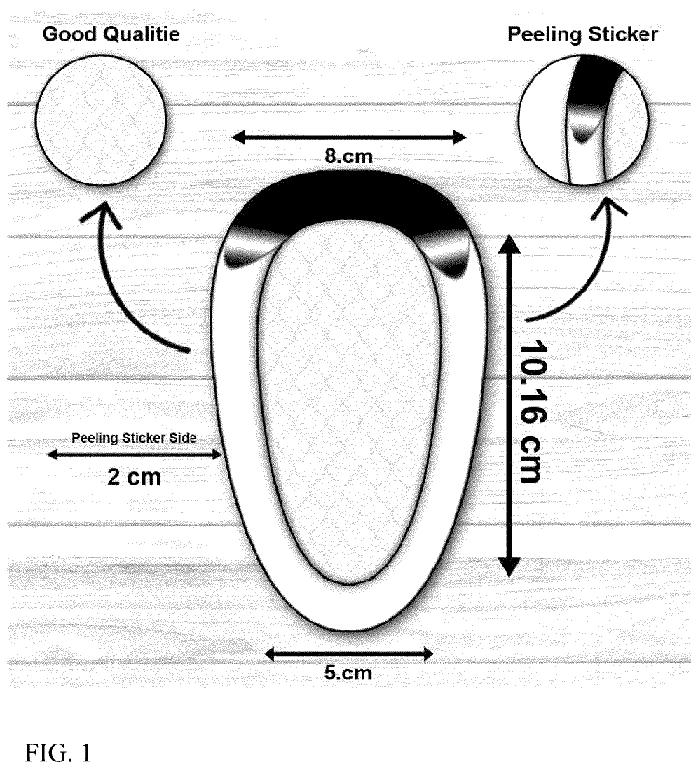

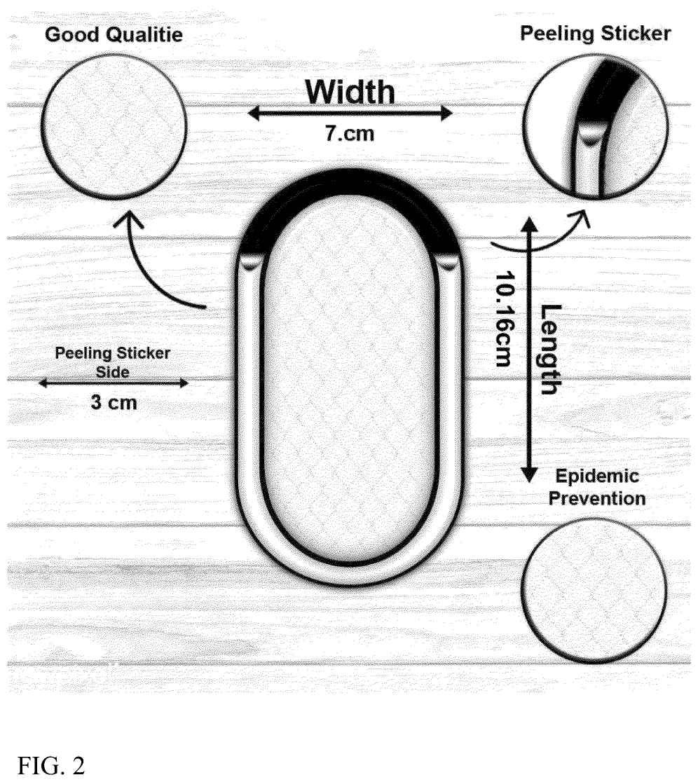

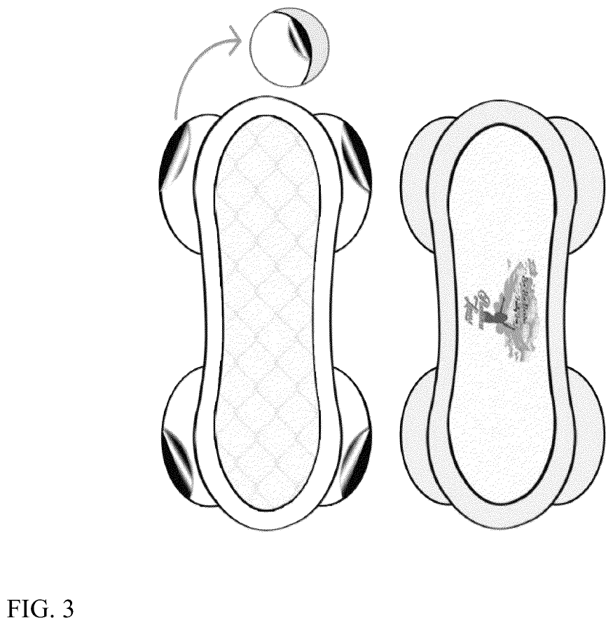

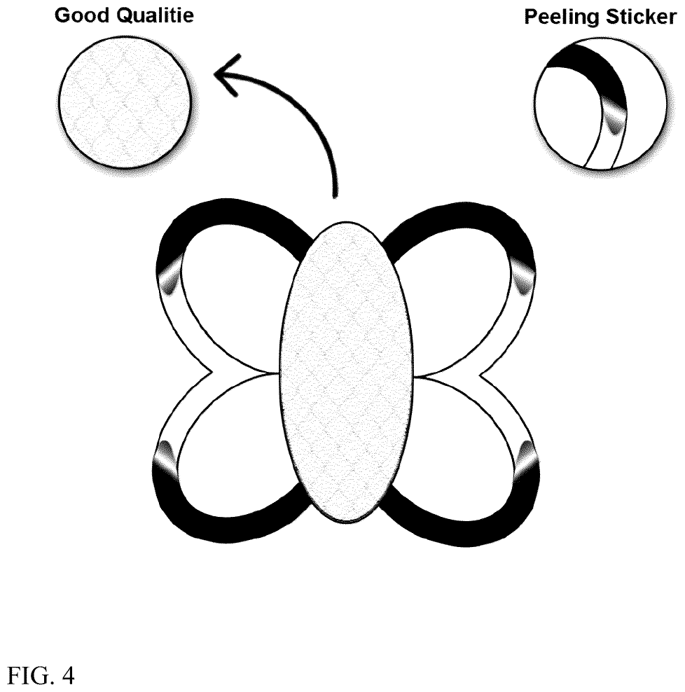

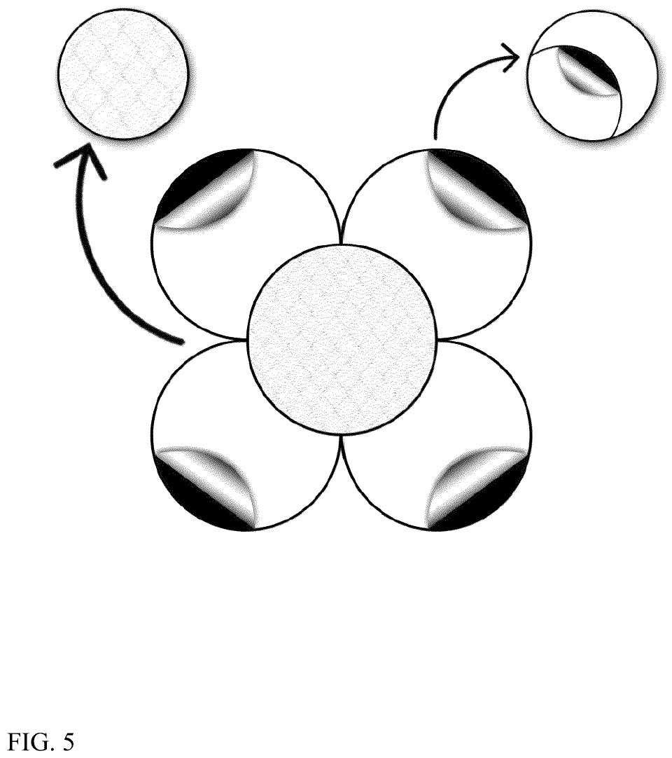

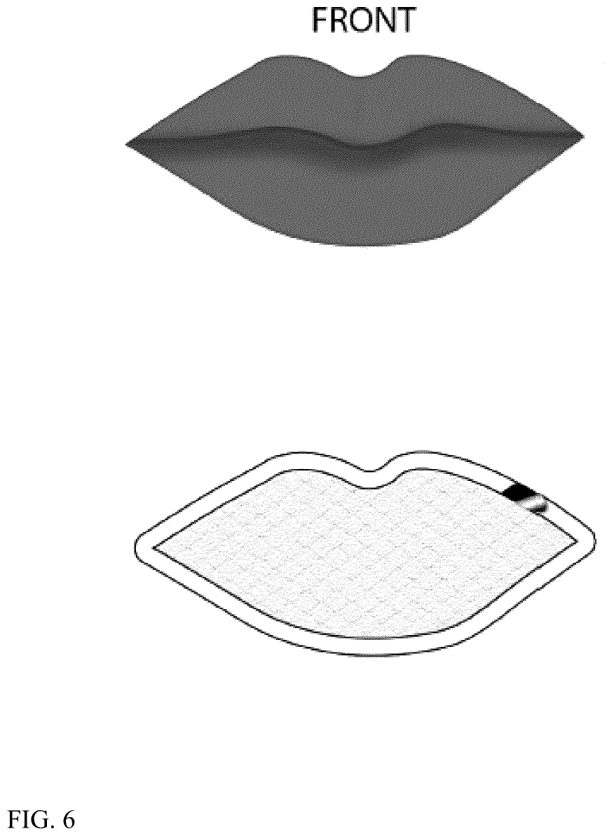

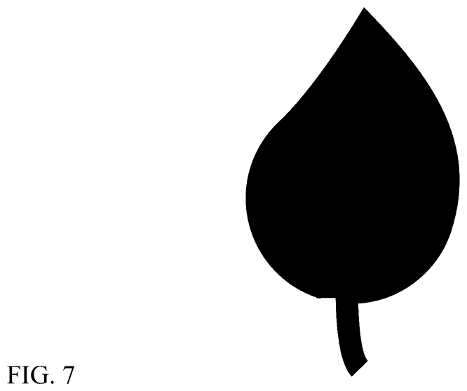

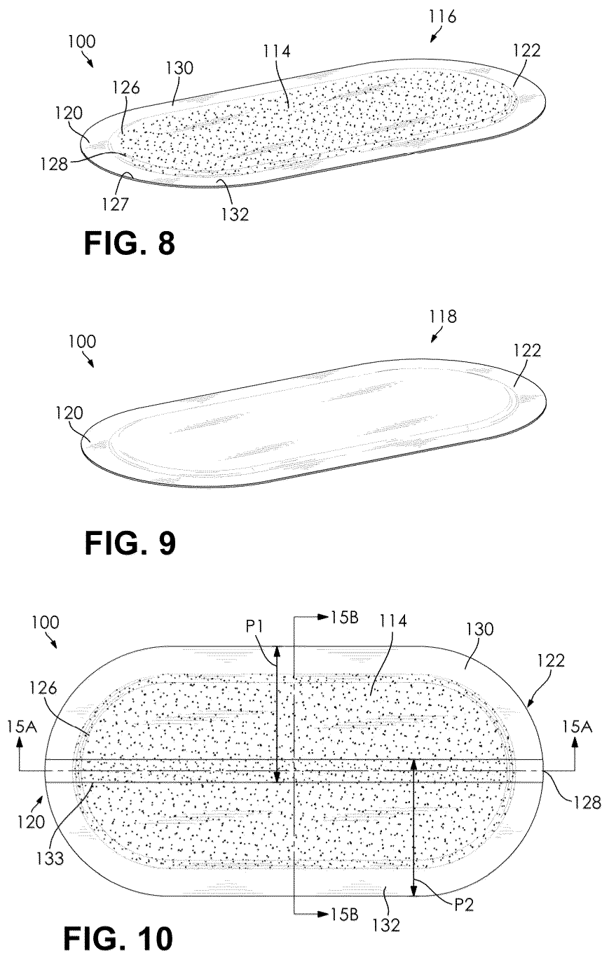

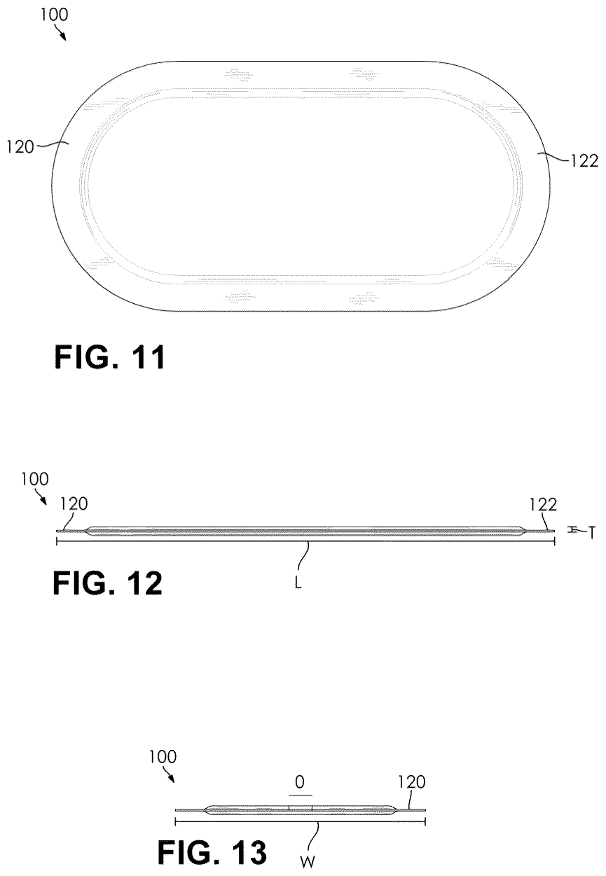

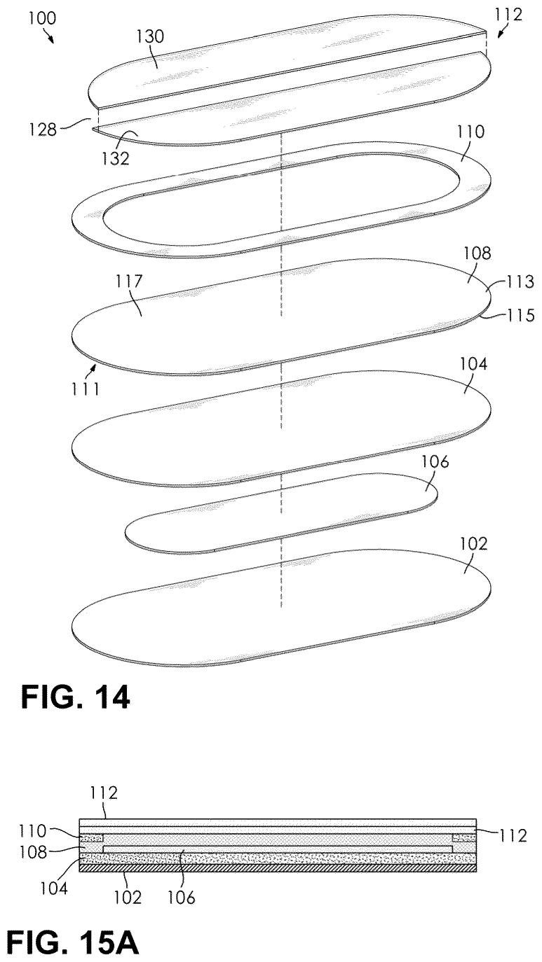

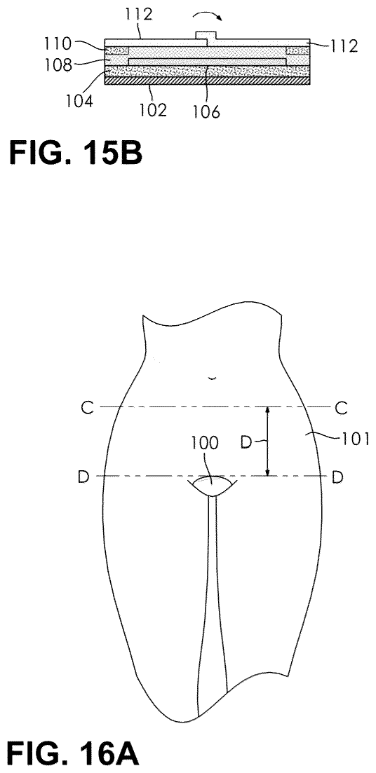

The present technology relates to a pantiless liner 100 for use without an undergarment by alleviating the need to use an undergarment, as shown generally in FIGS. 1-16. With particular reference to FIGS. 14-15, the pantiless liner 100 can include a moisture-impervious layer 102, a transfer adhesive layer 104, a super absorbent polymer (SAP) layer 106, a fabric layer 108, a body adhesive layer 110, and a protective layer 112. The SAP layer 106 and the fabric layer 108 together form a core 114 of the pantiless liner 100. The pantiless liner 100 can include a topside 116 and an underside 118, as shown in FIGS. 8-11. In operation, the topside 116 can be configured to rest against the body and in other words, the topside 116 can be bodyside. In operation, the underside 118 can contact the inner thigh of the user 101 as well as the article of clothing that the user 101 is wearing, such as leggings, jeans, pants, or slacks, as examples.

As shown in FIG. 1, the pantiless liner 100 can include a front portion 120 and a rear portion 122. In operation, the front portion 120 can be positioned slightly above the clitoral hood 105 of the user 101 such that the body adhesive layer 110 rests directly above the clitoral hood 105 on the external female anatomy and the fabric layer 108 is positioned on the clitoral hood 105. In other words, an inner boundary 126 between the body adhesive layer 110 and the fabric layer 108 rests substantially on a top edge of the clitoral hood 105 of the user 101. In operation, the rear portion 122 of the pantiless liner 100 can be positioned on a portion of the perineum 103 of the user 101.

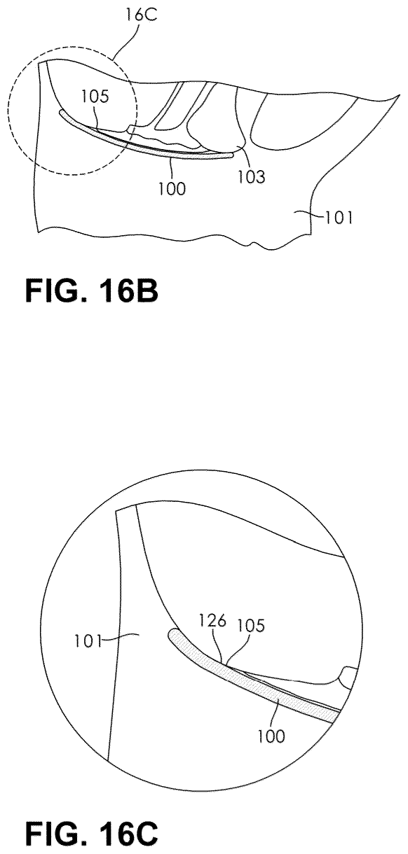

With reference to FIG. 14, the moisture-impervious layer 102 can be the bottom-most layer of the pantiless liner 100 such that an underside of the moisture-impervious layer 102 is the underside 118 of the pantiless liner 100 and faces away from the vulva 107 and vaginal opening, as shown in FIG. 16B-16C. The moisture-impervious layer 102 can be configured to be highly resistant to the passage of water and other bodily fluids, such as discharge and light menstrual spotting, to facilitate effective leakage protection. In this way, the moisture-impervious layer 102 can be configured to militate against moisture permeating the pantiless liner 100. The moisture-impervious layer 102 can include a low water vapor transmission rate (WVTR), which facilitates moisture not passing through the material, thereby providing a dry surface and preventing leakage.

One advantage of incorporating the moisture-impervious layer 102 in the pantiless liner 100 is to maintain a dry surface, which is important for comfort and skin health. By militating against moisture from seeping through, the moisture-impervious layer 102 can help to reduce the risk of skin irritation and infection, which can be caused by prolonged exposure to moisture. Additionally, the moisture-impervious layer 102 contributes to the overall structural integrity of the pantiless liner 100, militating against the pantiless liner 100 disintegrating or losing shape when wet, and thus promoting reliable performance during use. The moisture-impervious layer 102 can also provide a degree of flexibility and stretch, which is important for products that need to conform to the body's shape and movements without compromising their barrier function.

Additionally, the moisture-impervious layer 102 can allow for the pantiless liner 100 to provide discreet protection without bulkiness. The moisture-impervious layer 102 can be chemically inert and non-reactive, to militate against degradation or alteration when in contact with bodily fluids or the various materials used in hygiene products. Further, the moisture-impervious layer 102 can be non-toxic and hypoallergenic, minimizing the risk of skin irritation or adverse reactions. As a non-limiting example, the moisture-impervious layer 102 can be formed from materials such as polypropylene, polyethylene, polyvinyl chloride, polyurethane, polyester, as well as biodegradable films. These examples can be selected for their safety, durability, and comfort. In a preferred embodiment, the moisture-impervious layer 102 can be constructed from 0.02 mm watertight polyethylene film. A skilled artisan can select other suitable moisture-impervious materials for the moisture-impervious layer 102.

As shown in FIG. 14, the transfer adhesive layer 104 can be disposed between the moisture-impervious layer 102 and the SAP layer 106. Where the pantiless liner is assembled into a single product for use, the transfer adhesive layer 104 can be configured to laminate the moisture-impervious layer 102, the SAP layer 106, and the fabric layer 108 together.

During lamination, where the transfer adhesive layer 104 is disposed adjacent to the moisture-impervious layer 102, the SAP layer 106, and the fabric layer 108, and the layers 102, 104, 106, 108 are aligned, heat and pressure can be applied to the stacked layers to activate the transfer adhesive layer 104. In this way, a portion of the moisture-impervious layer 102 and the core 114 can be coupled. In certain embodiments, the portion of the moisture-impervious layer 102 and the core 114 that can be coupled is a border that is disposed along the same portion of the pantiless liner 100 as the body adhesive layer 110 and can correspond with this portion of the body adhesive layer 110, described herein below. As a non-limiting example, an exposable portion 117 of the fabric layer 108 can correspond with the portion of the moisture-impervious layer 102 and the core 114 that the transfer adhesive layer 104 is not disposed on.

During manufacturing, heat and pressure can be applied to bond the moisture-impervious layer 102, the SAP layer 106, and the fabric layer 108. This can result in improved strength and stability of the pantiless liner 100. The transfer adhesive layer 104 can allow for precise application of the adhesive found in the transfer adhesive layer 104, resulting in a clean, uniform bond line without the mess or variability that can come with liquid adhesives or hot melts. Advantageously, lamination can be highly efficient and is easily integrated into automated production lines, enhancing throughput and consistency in manufacturing.

Advantageously, using the transfer adhesive layer 104 and the process of lamination in the construction of the pantiless liner 100 allows for the combination of a wide range of materials, including those that are difficult to bond using traditional methods. The bonding process that occurs during lamination of the transfer adhesive layer 104 can be clean and requires no curing time, which accelerates the production process and reduces the need for additional drying or curing equipment. Moreover, the bond created by transfer tapes, such as the transfer adhesive layer 104, is often highly durable, resistant to aging, and can withstand various environmental conditions, making it suitable for products that are exposed to harsh environments or require long-term reliability. Additionally, the thickness of the transfer adhesive layer can be controlled to allow for a thinner pantiless liner 100 to be produced.

As a non-limiting example, a variety of polymers and agents can be employed depending on the specific requirements of the lamination process. As a non-limiting example, common adhesive polymers include acrylics, rubber-based adhesives, and silicone adhesives. In a specific embodiment, the transfer adhesive layer 104 can include an acrylic adhesive. In a preferred embodiment, the transfer adhesive layer 104 can be the Adhesive Transfer Tape 467MP manufactured by 3M. Advantageously, the 3M Adhesive Transfer Tape 467MP can be humidity resistant, UV resistance, water resistant, and temperature cycling resistant. A skilled artisan can select a suitable material for the transfer adhesive layer 104 within the scope of the present disclosure.

With continued reference to FIG. 14, the SAP layer 106 can be disposed between the transfer adhesive layer 104 and the fabric layer 108. As described above, the SAP layer 106 and the fabric layer 108 can form the core 114 of the pantiless liner 100. The core 114 of the pantiless liner 100 can act as the primary absorbent area. The function of the core 114 can be to quickly wick away moisture from the skin, trapping it within its structure to militate against any leakage or dampness on the surface closest to, or touching, the skin. The core 114 can be constructed of absorbent materials that are selected for their capillary action and fluid retention capabilities, allowing the pantiless liner 100 to remain thin, light weight, and discreet while still providing protection. The fibers or super absorbent polymers within the core 114 can be distributed in a gradient, with higher concentrations in areas where absorption is most needed, optimizing the effectiveness of the pantiless liner 100. Alternatively, the fibers or super absorbent polymers within the core can be distributed uniformly over the area of the core 114. The ability of the core 114 to absorb liquid quickly also minimizes any potential for odor, as the moisture is locked away from exposure to air.

In certain embodiments, the core 114 can be fabricated from a non-woven material, described herein as the fabric layer 108, as well as a super absorbent polymer, described herein as the SAP layer 106. As a non-limiting, the super absorbent polymer can be present in an amount of 15-20% of the core 114, while the non-woven makes up approximately 80-85% of the material of the core 114. A skilled artisan can select a suitable ratio of fluff material and super absorbent polymer within the scope of the present invention.

It should further be appreciated that the core 114 can be seamless and may or may not have stitching, hemming, bonding or other treatment along some or all of the periphery of the core such that the border of the core 114 and the moisture-impervious layer 102 are bonded, as described hereinabove. A skilled artisan can select a suitable securing means for the layers as desired.

The SAP layer 106 can include cross-linked polymers that are capable of absorbing and retaining extremely large amounts of a liquid relative to their own mass. The SAP layer 106 can absorb several times its weight in water, aqueous solution, or liquid and can swell to form a gel-like substance in the pantiless liner 100. The SAP layer 106 can achieve this due to the hydrophilic functional groups in the polymer chain, which interact with water molecules. The cross-linking within the polymer militates against the SAP layer 106 from dissolving in the water it absorbs, allowing SAP layer 106 to maintain its structural integrity even should the SAP layer 106 become fully saturated.

Advantageously, the SAP layer 106 can enhance the performance of the product by absorbing large volumes of liquid quickly, thus providing dryness and improved skin health. The SAP layer 106 can also help in reducing the overall weight and bulkiness of the product, leading to more comfort and discretion for the user 101. Several types of super absorbent polymers can be used for the SAP layer 106. As a non-limiting example, the SAP layer 106 can include sodium polyacrylate, a polyacrylamide copolymer, and a starch-based super absorbent polymers. A skilled artisan can select a suitable super absorbent polymer or any other type of polymer or super absorbent material suitable for the SAP layer 106 within the scope of the present disclosure.

As shown in FIG. 14, the fabric layer 108 can be a part of the core 114 and can be disposed between the SAP layer 106 and the body adhesive layer 110. The fabric layer 108 can include a lower surface 111, an upper surface 113, and a perimeter 115. The upper surface 113 can include an exposable portion 117.

Where the pantiless liner 100 is in use on the external female anatomy, the fabric layer 108 can form a portion of the topmost layer of the pantiless liner 100 such that the fabric faces upward and the exposable portion 117 contacts the vulva 107 and vaginal opening, as shown in FIGS. 16B and 16C. For this purpose, the fabric layer 108 provides a soft, cloth-like feel that is gentle against the skin. The fabric layer can be constructed from fibers that are bonded together by chemical, mechanical, heat, or solvent treatment, rather than being woven or knitted. Non-woven fabrics can be selected for the top layer of the pantiless liner 100 due to their breathability, softness, and ability to promote a healthy skin environment.

Advantageously, the non-woven fabrics in the pantiless liner 100 allows air to circulate, reducing the risk of moisture buildup and providing a comfortable and dry feel. Breathability is crucial for maintaining skin health, as it helps to militate against irritation and rashes that can occur from prolonged exposure to moisture. Non-woven fabrics can also be highly absorbent, quickly wicking away moisture from the skin and transferring it to the absorbent core 114 of the pantiless liner 100. This wicking action is facilitated by the capillary action of the fibers to draw liquid away from the surface and into the core 114.

As a non-limiting example, the non-woven fabric of the fabric layer 108 can include polypropylene, polyester, or viscose. These synthetic materials can be selected for their strength, durability, and ability to be engineered with specific properties, such as softness and absorbency. In some cases, biodegradable and natural fibers, such as cotton or bamboo, can be incorporated to enhance the comfort and environmental friendliness of the product. In certain embodiments, the fabric layer 108 includes a single layer of non-woven fabric. Optionally, the pantiless liner 100 can include multiple layers of non-woven fabric, depending on the amount of absorbency required by the user 101.

The fabric layer 108 can be flexible or form retaining. Where the core 114 includes multiple fabric layers 108, the each of the multiple layers can be formed of any such materials mentioned here and can be formed of the same or different materials. In certain embodiments, the core 114 can be sufficiently stretchable and malleable to take the shape of, in use, the vulva 107 of the wearer and comfortably form to the wearer.

The surface of the fabric layer 108 can be treated with various finishes or embossed with patterns to improve the feel against the skin and to aid in fluid management. In certain embodiments, the fabric layer 108 can be embossed with a pattern configured to move fluids through and across the fabric layer 108 and the SAP layer 106, in operation. The pattern can also improve an overall softness of the fabric layer 108, which is beneficial due to the proximity of the SAP layer 106 to the user 101, in operation. In one non-limiting example, the embossed pattern can include depressions along the surface area of the core 114.

It should be appreciated that the fabric layer 108, including the non-woven fabric itself, does not need to be the primary absorbent layer. Rather, the fabric layer 108 can act as a transfer layer that quickly passes fluids to the absorbent SAP layer 106 beneath. The fibers in the non-woven fabric are designed to spread the liquid over a wide area, preventing pooling and promoting faster absorption by the SAP layer 106. This distribution is essential for militating against leaks and ensuring that the panty liner can handle sudden surges of liquid.

Additionally, with respect to the interaction of the fabric layer 108 with the skin, non-woven fabrics can be hypoallergenic and non-irritating. The softness of the material can militate against friction and provide a comfortable experience, even for those with sensitive skin. Additionally, the structure of the non-woven fabric can militate against linting or pilling, which can otherwise cause discomfort.

The length of the fabric layer 108 determines the core 114 and, as such, is related to the absorbance of the pantiless liner 100. In certain embodiments, the length of the fabric layer 108 can be between about 3 inches and about 5 inches, as an example. In a more particular embodiment, the length of the fabric layer 108 can be between about 4.2 inches and about 4.2 inches, as an example. In a most particular embodiment, the length of the fabric layer 108 can be about 4 inches, as an example. The A skill artisan can select a suitable length of the pantiless liner 100 within the scope of the present disclosure. Desirably, limiting the pantiless liner 100 to this length limits the applicable position of the pantiless liner 100 requiring the user 101 to only position the pantiless liner 100 between the clitoral hood 105 and the perineum 103.

It should be appreciated that FIGS. 15A-15B are schematic representations of the location of the layers of the pantiless liner 100. The representations of FIGS. 15A and 15B are not representative of the thickness or rigidity of the layers.

As shown in FIGS. 15A-15B, the body adhesive layer 110 can be disposed on the upper surface 113 of the fabric layer 108. The body adhesive layer 110 can be disposed adjacent the perimeter 115 of the fabric layer 108. The body adhesive layer 110 can include an inner boundary 126 and an outer boundary 127. The outer boundary 127 can be coextensive with the perimeter 115 of the fabric layer 108 such that the outer boundary 127 and the perimeter 115 can be coplanar. The inner boundary 126 can circumscribe the exposable portion 117 of the upper surface 113 of the fabric layer 108 and result in the body adhesive layer 110 appearing as a border placed on the fabric layer 108. In this way, the body adhesive layer 110 can abut the upper surface 113 of the fabric layer 108. The body adhesive layer 110 can be disposed adjacent to the protective layer 112 while the protective layer 112 is in use.

In operation, the body adhesive layer 110 can be configured to contact and adhere to the vulva 107 and perineum 103 of the user 101 in an effort to close off the vaginal opening with the pantiless liner 100. The body adhesive layer 110 can be a dual-layer adhesive layer in certain embodiments and can be configured to be repositioned by the user after initial application without loss of adhesive properties.

The body adhesive layer 110 can be constructed from silicone. Advantageously, silicone is hypoallergenic, chemical stable, and flexible. In this way, silicone is generally non-irritating and can be used on a wide range of skin types. Additionally, silicone adhesives can be resistant to moisture and body fluids, facilitating that the panty liner remains securely in place throughout the day, regardless of humidity levels or perspiration. The resistance to moisture also means that the adhesive should not break down or become less effective when exposed to the conditions typically found in the intimate environment.

Additionally, the body adhesive layer 110 can be constructed from acrylic. The acrylic adhesive can be a synthetic resin. Advantageously, acrylic adhesives provide a strong, durable bond and are known for their long-term adhesion properties. Acrylic adhesives also have good resistance to environmental factors such as heat and moisture, which is essential for products that are subject to body heat and fluids. The resistance to degradation over time means that the adhesive will maintain its bonding strength, militating against the pantiless liner 100 from shifting or bunching. Another advantage of acrylic adhesives is their versatility in terms of formulation. They can provide a range of adhesive strengths, from very light to very strong, allowing the body adhesive layer 110 to be tailored to the specific needs of the pantiless liner 100. Acrylic adhesives can also be formulated to minimize the potential for skin irritation, making them suitable for use in products that come into contact with sensitive skin areas.

As described above, the body adhesive layer 110 can be a border that circumscribes the fabric layer 108. The body adhesive layer 110 can include the inner boundary 126 where the body adhesive layer 110 meets the fabric layer 108. In operation, the inner boundary 126 can be positioned on the user 101 adject the clitoral hood 105 such that the body adhesive layer 110 rests on the vulva 107 directly above the clitoral hood 105. It should also be appreciated that the SAP layer 106 can be confined within the exposable portion 117 of the fabric layer 108 and within the distance of the border (B) created by the body adhesive layer 110, as shown in FIG. 15B.

The body adhesive layer 110 can have a uniform width, as shown in FIG. 10. As a non-limiting example, the body adhesive layer 110 can include a width between about 0.3 inches and about 0.5 inches. In a more particular embodiment, the body adhesive layer 110 can include a width between about 0.35 inches and about 0.45 inches. In a most particular embodiment, the body adhesive layer 110 can include a width of about 0.39 inches. Advantageously, a skilled artisan can select a suitable width for the body adhesive layer 110 within the scope of the present disclosure.

It should be appreciated that applying the adhesive only along the border of the panty liner brings its own set of advantages. By restricting the adhesive to the border, the exposable portion 117 remains free of any sticky substances, which can enhance the breathability of the pantiless liner 100 and contribute to a more comfortable experience for the user 101. Further, this can militate against the potential for irritation caused by adhesive contact with the skin and can militate against the feeling of tackiness that can sometimes be associated with adhesive products.

Furthermore, having the body adhesive layer 110 only on the border of the fabric layer 108 allows for easier adjustability and repositioning of the panty liner. Users 101 can reposition the liner for a better fit without dealing with the entire adhesive surface, which can sometimes cause the liner to wrinkle or fold. This adjustability is particularly beneficial for users 101 who are active or may need to reposition their liners throughout the day for optimal comfort and protection. The placement of body adhesive layer 110 on the border of the fabric layer 108 can also simplify the manufacturing process, as it requires less adhesive material and reduces the risk of adhesive contaminating the core 114 during production. This can lead to cost savings for manufacturers and potentially lower prices for consumers.

The body adhesive layer 110 can be on the pantiless liner 100 as packaged, such that it is ready to be worn. In certain embodiments, the body adhesive layer 110 can be a bilayer including a first sublayer and a second sublayer. The first sublayer and the second sublayer together can make up the body adhesive layer 110. The use of the first sublayer and the second sublayer can provide a stronger and more reusable body adhesive layer 110. For example, the first sublayer along the perimeter of the fabric layer 108 can be stronger or less strong than a second sublayer. Suitable combinations of two or more adhesives can be selected by one of ordinary skill in light of the present disclosure. Similarly, any suitable one or more pre-applied adhesive and/or inherently adhesive material may be used in combination with suction of the web and/or adherent grooves, ditches, imprints, ridges, or other adherent applications of the web to the wearer, as discussed elsewhere in this disclosure.

Suitable silicone adhesives can be selected by one of those skilled in the art. The first sublayer can be an acrylic adhesive such as the Medical Tape, Product Number 1522, manufactured by 3M, for example. The second sublayer can be silicone such as Thermoplastic Elastomer (TPE) Tape with Silicone Adhesive, Product Number 2475, manufactured by 3M, for example. A skilled artisan can select a suitable acrylic adhesive, silicone adhesive, or combination thereof within the scope of the present disclosure.

The body adhesive layer 110 can be pre-applied to the border of the fabric layer 108 of the body side of the pantiless liner 100. With such pre-applied adhesive, the undergarment in certain exemplary embodiments can be applied onto the body by the wearer or an assistant after removal of the protective layer 112. As described hereinbelow, the body adhesive layer 110 can be a pressure sensitive or contact adhesive, such that the pantiless liner 100 can be applied by positioning it and applying gentle pressure. As a non-limiting example, the adhesive can be heat activated to allow for the pantiless liner 100 to be applied by holding it in position on the vulva 107 and perineum 103 for a short time such that the undergarment can be applied by holding it in position for a short time to allow for body heat to activate the silicone adhesive.

The body adhesive layer 110 can be uniform or non-uniform in thickness over the fabric layer 108. Optionally, the body adhesive layer 110 can differ in thickness from one area of the pantiless liner 100 to another. For example, the body adhesive layer 110 can be applied to the fabric layer 108 in a greater thickness at the lateral ends of the pantiless liner 100 in the areas that adhere to the perineum 103 or portions of the perineum 103 of the user 101 to account for the increases concentration of moisture found at the vaginal opening, adjacent the perineum 103. Further, in certain embodiments different silicone adhesives can be used in different portions of the undergarment. As a non-limiting example, a less aggressive adhesive that forms a weaker bond to skin, can be used in direct contact with the vulva 107 or portions of the vulva 107, while more aggressive adhesive that forms a stronger adhesive bond to skin, can be used in contact with the perineum 103 or portions of the perineum 103.

With reference to FIGS. 14-15B, the protective layer 112 can be the uppermost layer of the pantiless liner 100 before it is removed by the user 101 to expose the body adhesive layer 110 and the fabric layer 108. The protective layer 112 can selectively cover the exposable portion 117 of the upper surface 113 of the fabric layer 108 and be removably adhered to the fabric layer 108 by the body adhesive layer 110. The protective layer 112 can include removable film or sheet. The protective layer 112 can cover the entirety of the pantiless liner 100 including the body adhesive layer 110 and the fabric layer 108. Although the protective layer 112 can cover all of the body adhesive layer 110 and the fabric layer 108, the protective layer 112 can be adhered to only the body adhesive layer 110 to allow for easy removal of the protective layer 112 in operation.

The removable protective layer 112 can be used with the pantiless liner 100 for user 101 convenience and hygiene in mind. The protective layer 112 can serve as a removable cover that can shield the body adhesive layer 110 and the fabric layer 108 until the pantiless liner 100 is ready to be used. To facilitate easy removal, the protective layer 112 can include a split 128 disposed lengthwise, resulting in an overlap (O) that allows the user 101 to peel the protective layer 112 away where the user 101 is ready to use the pantiless liner 100. The split 128 can result in the protective layer having a first panel 130 and a second panel 132 that overlap. The first panel 130 can overlap the second panel 132. In an alternative embodiment, the split 128 can be widthwise along the protective layer 112. The first panel 130 can have a first width (P1) and the second panel 132 can have a second width (P2). The first width (P1) can be greater than the second width (P2). The first panel 130 can include a terminal edge 133 that overlaps the second panel 132 and is configured to assist the user with removal of the protective layer from the pantiless liner.

In certain embodiments, the overlap of the first panel 130 and the second panel 132 on the protective layer 112 can be between about 0.1 inches and about 0.3 inches, as an example. In a more particular embodiment, the overlap of material on the protective layer 112 can be between about 0.15 inches and about 0.25 inches, as an example. In a most particular embodiment, the overlap of material on the protective layer 112 can be about 0.2 inches, as an example. A skill artisan can select a suitable overlap of the material on the protective layer 112 within the scope of the present disclosure.

This aspect of the protective layer 112 allows for the process of applying the panty liner to be straightforward and militate against hassle during application, which is particularly appreciated during quick changes or when using the product on the go. The overlap of the protective layer 112 along the split 128 area also militates against the body adhesive layer 110 from being exposed prematurely, which could cause the liner to stick to itself or other surfaces accidentally. The protective layer 112 assists with the body adhesive layer 110 remaining covered and clean until the moment of application, preserving its stickiness and effectiveness. Additionally, the protective layer 112 helps to maintain the sanitation and sterility of the fabric layer 108. Primarily, the protective layer acts as a barrier against contaminants such as dust, dirt, and moisture, which is crucial for a product that is often carried in handbags or pockets and stored in bathrooms, where it may be exposed to various environmental factors. During the manufacturing process, the pantiless liner 100 can be produced in a controlled environment to militate against harmful bacteria and other pathogens from embedding into the pantiless liner 100. The protective layer 112 helps maintain this sterility by sealing off the body side, including the body adhesive layer 110 and the fabric layer 108, which is the part of the pantiless liner 100 that can comes into contact with the skin of the user 101, particularly the vulva 107 and perineum 103 of the user 101.

Suitable materials for the protective layer 112 can be selected by one of those skilled in the art. The protective layer 112 can be constructed from plastic. As a non-limiting example, the protective layer 112 can be formed from polyester, polyethylene, polycarbonate, polypropylene, acrylic, polyvinyl chloride, or combinations thereof. In a preferred embodiment, the protective layer 112 can be formed from biaxially-oriented polyethylene terephthalate (BoPET), a polyester film made from stretched polyethylene terephthalate (PET).

With reference to FIG. 16A, the pantiless liner 100 can be shaped to minimally cover the vulva 107, vaginal opening, and a portion of the perineum 103 of the user 101. Specifically, the pantiless liner 100 of the present disclosure is shaped, and sized, to cover the clitoral hood 105, clitoris, labia majora, urethral opening, vaginal opening, and a portion of the perineum 103. Accordingly, the pantiless liner 100 does not extend onto the mons pubis or the anus of the user 101. In operation, the body adhesive layer 110 can be placed directly above the clitoral hood 105, as shown in FIGS. 16B-16C, the exterior of the labia majora, and the perineum 103. In this way, and as shown in FIG. 16B, the clitoral hood 105, clitoris, labia majora, urethral opening, vaginal opening, and a portion of the perineum 103 are covered by the pantiless liner 100. Such embodiments are shaped and sized to cover the vulva 107 without extending forward of the clitoral hood 105. The precise positioning of the pantiless liner 100 on the external female anatomy militates against the pantiless liner 100 from contacting the mons pubis of the user 101 and, therefore, militate against friction between the pantiless liner 100 and as the article of clothing that the user 101 is wearing, such as leggings, jeans, pants, or slacks, as examples.

As described herein, the pantiless liner 100 can be pre-configured to form to the vulva 107 of the wearer. In certain embodiments the undergarment can be packaged in a substantially flat condition and where the pantiless liner 100 is removed from the packaging and positioned on the user 101, assumes a different shape. It should also be appreciated that the pantiless liner 100 can be an egg shape, an oval shape, a butterfly shape, a flower shape, lips shape, and a leaf shape, as shown in FIGS. 1-8.

As described hereinabove, the pantiless liner 100 can be sized to cover the clitoral hood 105, clitoris, labia majora, urethral opening, vaginal opening, and a portion of the perineum 103 and the front portion 120 can be disposed Axis D, as shown in FIG. 16A. Accordingly, the pantiless liner 100 does not extend onto the mons pubis or the anus of the user 101. As shown in FIG. 16A, there can be a distance (D) between the bikini line, at Axis C, and the position of the front portion 120 of the pantiless liner at Axis D.

With reference to FIG. 16A, the pantiless liner 100 does not approach the bikini line, shown as Axis C, of the user 101 and is positioned much further down on the female anatomy. In a particular embodiment, the pantiless liner 100 can include a length (L), a width (W), and a thickness (T). The length (L) of the pantiless liner 100 determines the coverage area, as described above, the pantiless liner is sized to extend between the clitoral hood 105 and a portion of the perineum 103. In certain embodiments, the length (L) of the pantiless liner 100 can be between about 4.2 inches and about 5.2 inches, as an example. In a more particular embodiment, the length (L) of the pantiless liner 100 can be between about 4.5 inches and about 4.9 inches, as an example. In a most particular embodiment, the length (L) of the pantiless liner 100 can be about 4.7 inches, as an example. A skill artisan can select a suitable length of the pantiless liner 100 within the scope of the present disclosure and such that the pantiless liner 100 covers only the clitoral hood 105, clitoris, labia majora, urethral opening, vaginal opening, and a portion of the perineum 103. Desirably, limiting the pantiless liner 100 to this length limits the applicable position of the pantiless liner 100 requiring the user 101 to only position the pantiless liner 100 between the clitoral hood 105 and the perineum 103.

The width of the pantiless liner 100 determines coverage of the pantiless liner 100 in the space between the thighs of the user 101, specifically between the inner thigh portion of the user 101, and more specifically, the distance between the exterior of the labia majora. In certain embodiments, the width (W) of the pantiless liner 100 can be between about 2 inches and about 3 inches, as an example. In a more particular embodiment, the width (W) of the pantiless liner 100 can be between about 2.3 inches and about 2.7 inches, as an example. In a most particular embodiment, the width (W) of the pantiless liner 100 can be about 2.5 inches, as an example. A skill artisan can select a suitable width for the pantiless liner 100 within the scope of the present disclosure.

The thickness of the pantiless liner 100 is indicative of the absorbency level and the overall feel of the product against the skin. In certain embodiments, the thickness (T) of the pantiless liner 100 can be between about 0.75 millimeters and about 1.25 millimeters, as an example. In a more particular embodiment, the thickness (T) of the pantiless liner 100 can be between about 0.9 millimeters and about 1.1 millimeters, as an example. In a most particular embodiment, the thickness (T) of the pantiless liner 100 can be about 1 millimeter, as an example. A skill artisan can select a suitable width for the pantiless liner 100 within the scope of the present disclosure.

In certain embodiment, the pantiless liner can include a thermo-responsive layer. The thermo-responsive layer can enhance user comfort, particularly for those leading active lifestyles or living in warmer climates. The thermo-responsive layer can be configured to react to body temperature changes, providing a cooling effect when the body overheats. One such thermos-responsive layer that could be integrated into the pantiless liner 100 is a cooling gel layer. The cooling gel could be activated by body heat and can be configured to maintain a temperature slightly below body temperature, thus offering a cooling sensation. The cooling gel could be encapsulated in microbeads or a thin layer within the core of the pantiless liner 100. Advantageously, the thermos-responsive can be beneficial during exercise, outdoor activities, or any situation where heat discomfort is a concern, helping to maintain comfort and prevent irritation.

Suitable materials for the thermo-responsive layer include materials such as phase change materials (PCMs) or hydrogels. Hydrogels are highly absorbent polymers that can retain a significant amount of water. When incorporated into the pantiless liner 100, these hydrogels could provide a cooling effect as the water slowly evaporates in response to increased body temperature. Both PCMs and hydrogels are suitable for use against the skin and can be integrated into the pantiless liner 100 in a way that does not compromise the thin and flexible nature of the pantiless liner 100.

The integration of organic and biodegradable materials into the pantiless liner 100 represents a significant step towards environmental sustainability and user health consciousness. By utilizing organic materials such as cotton, bamboo fibers, or other plant-based textiles for the fabric layer, the pantiless liner 100 can offer a natural and hypoallergenic surface that is gentle on the skin. These organic materials are cultivated without the use of synthetic pesticides or fertilizers, reducing potential irritants and allergens. Moreover, the organic nature of these fibers ensures that they are breathable and soft, enhancing the comfort for the user. To further the eco-friendly aspect, the super absorbent polymer (SAP) layer 106 could be made from biodegradable materials such as biopolymers derived from renewable resources. These biopolymers are designed to break down more quickly and safely in the environment compared to traditional SAPs.

In addition to the organic fabric and biodegradable SAP layer 106, the moisture-impervious layer 102 and the adhesive components of the pantiless liner 100 could be developed from biodegradable films and adhesives. Materials such as polylactic acid (PLA), which is derived from fermented plant starch (often from corn), could serve as an effective moisture barrier while being fully compostable. Similarly, adhesives based on natural rubber or bio-based tackifiers could provide the necessary adherence properties without leaving a lasting footprint on the environment. By embracing organic and biodegradable materials, the pantiless liner 100 not only meets the needs of environmentally aware consumers but also sets a precedent in the personal care industry for sustainable product development.

In certain embodiments, the pantiless liner 100 can further include edge softening features to enhance user comfort, especially during extended periods of wear. Softening the edges of the pantiless liner 100 can militate against any potential chafing or discomfort that might arise from friction against the sensitive skin of the vulva and perineum. This can be achieved by using softer, more flexible materials along the edges or by employing a specialized edge-finishing technique that rounds off or pads the edges. For instance, the perimeter of the fabric layer 108, where the body adhesive layer 110 is applied, could be treated with a softening agent or constructed with a cushioning layer that gently transitions into the adhesive portion. Additionally, the moisture-impervious layer 102 could be designed with tapered edges to reduce any sensation of bulkiness. By prioritizing the softness of the edges, the pantiless liner 100 can offer a more seamless and irritation-free experience, allowing users to feel comfortable and secure throughout the day, regardless of their level of activity or attire.

In certain embodiments, the pantiless liner 100 can include variable absorbency zones within both the fabric layer 108 and the SAP (super absorbent polymer) layer 106. The absorbency of these areas can be tailored to fit the anatomical needs of the user, providing more absorption where it is most required while maintaining a slim and comfortable profile. In the fabric layer 108, which is in direct contact with the skin, the non-woven material could be engineered with varying fiber densities or weaves in specific zones. For example, the area corresponding to the vaginal opening, where discharge or spotting is likely to be heaviest, could feature a tighter weave or a blend of fibers with higher wicking capabilities. This would allow for rapid transfer of moisture away from the skin and into the absorbent core. Conversely, the zones that align with less discharge-prone areas could be designed with a looser weave, enhancing breathability and reducing material usage without compromising protection.

Similarly, the SAP layer 106 can include variable concentrations of absorbent polymers. Strategic placement of higher concentrations of SAP in the central region of the pantiless liner can aid the core in absorbing and retaining a larger volume of fluid or moisture where likely to occur. This targeted approach not only improves the efficiency of the pantiless liner 100 but also contributes to a thinner overall product, as the peripheral areas of the SAP layer 106 can be constructed with less absorbent material, reducing bulk and enhancing comfort.

In certain embodiments, the body adhesive layer 110 can include a strength indicator configured to provide a visual cue to the user about the adhesive's effectiveness over time. By incorporating a color-changing element into the adhesive formulation, the pantiless liner 100 can signal when the adhesive strength is diminishing and when it might be time to replace the liner. To achieve this effect, a safe, skin-friendly dye within the body adhesive layer 110 that reacts to changes in pH, temperature, or exposure to moisture—factors that could affect adhesive performance. For instance, the adhesive might initially present a certain color, such as a light blue, indicating a strong adhesive bond. Over time, as the adhesive comes into contact with body fluids and begins to lose its tackiness, the color could gradually shift to a different shade, such as pink, alerting the user that the liner's adherence is weakening. This visual indicator would be particularly useful for active users or those in situations where they cannot immediately feel a shift in the liner's position.

The introduction of a color-changing adhesive can not only be practical but also reassuring for users who want to ensure they have reliable protection throughout the day. It can militate against guesswork and provide a clear, easily interpretable sign that it is time to change the liner, thus maintaining hygiene and comfort. This feature could be especially beneficial for those with busy lifestyles, individuals with sensory impairments, or users who are new to using pantiless liners and may not yet be familiar with judging when to replace them.

In certain embodiments, the pantiless liner 100 can integrate health monitoring capabilities, offering users not just comfort and protection but also valuable insights into their personal health. By embedding pH indicators within the fabric layer 108 or the SAP layer 106, the pantiless liner 100 can serve as an early detection system for potential health issues. Vaginal pH is a key indicator of vaginal health, with normal levels typically ranging from 3.8 to 4.5. Deviations from this range could signal infections such as bacterial vaginosis or yeast infections. The pH indicators could be designed to change color when exposed to pH levels outside the normal range, providing a visual alert to the user. This immediate feedback can empower users to seek medical advice promptly, potentially leading to earlier intervention and treatment.

In addition to pH monitoring, the pantiless liner 100 can incorporate temperature sensors that track changes in body temperature, which can be another important health metric. Slight elevations in temperature could indicate the onset of infection or other health concerns. These sensors can be made from non-invasive, skin-safe materials that react to temperature fluctuations. The data collected by these sensors could be read visually on the pantiless liner 100 itself or potentially synced with a mobile app, allowing users to monitor their temperature over time and detect any unusual patterns. This level of health monitoring can transform the pantiless liner from a single-use hygiene product into a multi-functional health tool, providing users with peace of mind and encouraging proactive health management.

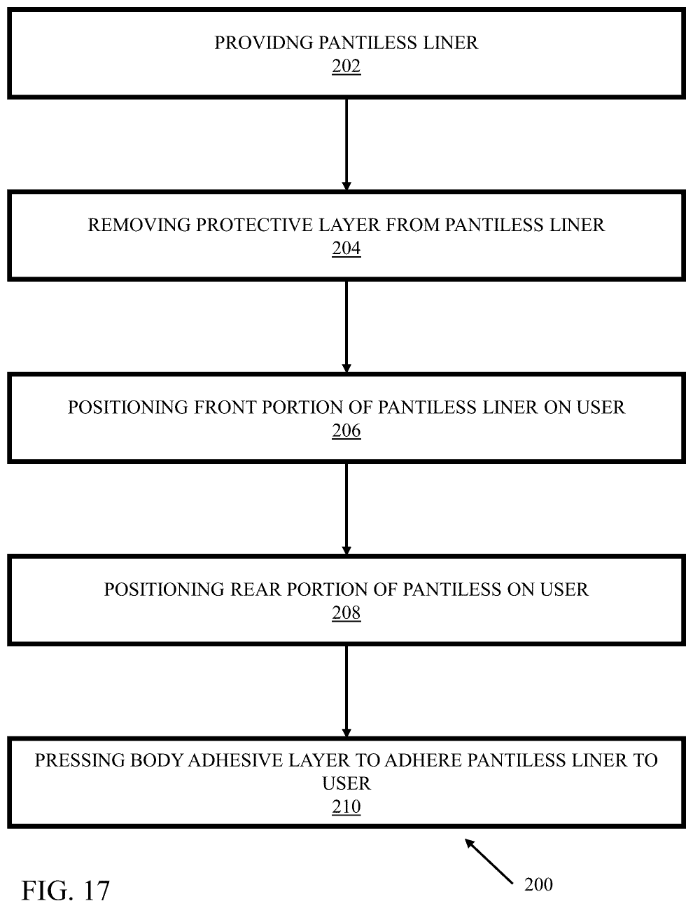

The present disclosure can further provide a method 200 for wearing a panty liner without an undergarment using a pantiless liner, as described hereinabove, and shown generally in FIG. 17. The method 200 can include a step 202 of providing the pantiless liner 100 as described hereinabove. The method 200 can include a step 204 of removing the protective layer 112 from the pantiless liner. As described hereinabove, the protective layer 112 can include a split 128 where portions of the protective layer 112 overlap to allow for the user 101 to easily remove the protective layer 112. The user 101 can lift the first panel 130 of the protective layer 112 and remove the first panel 130 from the body adhesive layer 110. The user 101 can use a gap that is created by removing the first panel 130 to lift the second panel 132 of the protective layer and remove the second panel 132 of the protective layer 112 from the body adhesive layer 110.

The method 200 can include a step 206 of positioning the pantiless liner 100 positioning the front portion 120 of the pantiless liner 100 such that the inner boundary 126 of the body adhesive layer 110 is positioned adjacent a clitoral hood 105 of user 101, as shown in FIG. 16C. As described hereinabove, the body adhesive layer 110 can include an inner boundary 126 where the body adhesive layer 110 and the fabric layer 108 meet. The inner boundary 126 can be positioned on the vulva 107 of the user 101 immediately above the clitoral hood 105. The precise positioning of the pantiless liner 100 on the external female anatomy militates against the pantiless liner 100 from contacting the mons pubis of the user 101 and, therefore, militate against friction between the pantiless liner 100 and as the article of clothing that the user 101 is wearing, such as leggings, jeans, pants, or slacks, as examples. This positioning can further militate against pubic hair of the user 101 becoming stuck in the body adhesive layer 110. By further militating against this friction force, the likelihood of the pantiless liner 100 becoming dislodged from or moving about the external female anatomy can be reduced.

The method 200 can include a step 208 of positioning the rear portion 122 the pantiless liner 100 such that the body adhesive layer 110 of the pantiless liner 100 is positioned on a portion of a perineum 103 of the user 101. The method 200 can also include a step 210 pressing the body adhesive layer 110 against the user 101 whereby the pantiless liner 100 is adhered to the user 101.

The method 200 can include a step 212 of laminating the transfer adhesive layer 104 to the portion of the moisture-impervious layer 102 and the core 114 via during a manufacturing process.

### EXAMPLES

Example embodiments of the present technology are provided with reference to the several figures enclosed herewith.

A first example describes where the user has an active lifestyle. The user is a fitness enthusiast who enjoys wearing leggings and prefers not to wear traditional undergarments during her workouts to avoid visible panty lines and discomfort. However, she wants to feel fresh and protected from vaginal discharge and light spotting that can sometimes occur.

The user takes a pantiless liner from its individual sterile packaging before heading to the gym. She removes the protective layer to expose the body adhesive layer. She positions the front portion of the pantiless liner so that the inner boundary of the body adhesive layer is adjacent to her clitoral hood. She then positions the rear portion of the pantiless liner so that it adheres to a portion of her perineum. After pressing the body adhesive layer against her skin to secure the pantiless liner, the user puts on her leggings and feels confident that the liner will stay in place and provide the necessary absorption during her workout. Post-workout, the user can easily remove the pantiless liner, which leaves no residue, and dispose of the pantiless liner.

A second example describes where the user has a fashion-forward event. The user has a gala event to attend and has chosen a form-fitting evening gown that requires a seamless look without the lines of traditional undergarments. She also wants to feel secure and clean throughout the long event, which includes a dinner and dance.

The user prepares for the event by selecting the pantiless liner that matches her needs for the evening. In the privacy of her home, she removes the protective layer from the pantiless liner, revealing the body adhesive. Carefully, she positions the pantiless liner, ensuring the front portion's inner boundary is just above her clitoral hood and the rear portion is on her perineum. She presses the adhesive gently against her skin, securing the pantiless liner in place. The user then dons her evening gown, enjoying a sleek silhouette without worrying about undergarment lines or discomfort. Throughout the event, the pantiless liner absorbs any moisture or discharge, keeping the user feeling dry and comfortable. After the event, she can remove the liner without any hassle and dispose of the pantiless liner.

In both examples, the pantiless liner provides a discreet, comfortable, and practical solution for women seeking an alternative to traditional undergarments while engaging in various activities.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A method for wearing a pantiless liner without an undergarment by a user, comprising steps of:
providing the pantiless liner having a front portion and a rear portion, the pantiless liner including
a moisture-impervious layer being a bottom-most layer of the pantiless liner,
a core including
a super absorbent polymer (SAP) layer including cross-linked polymers configured to absorb moisture or liquid, and
a fabric layer disposed adjacent to the SAP layer and including a non-woven material having a lower surface, an upper surface with an exposable portion, and a perimeter,
a transfer adhesive layer disposed between the moisture-impervious layer and the core and coupling a portion of the moisture-impervious layer to the core,
a body adhesive layer disposed on the upper surface and adjacent the perimeter of the fabric layer of the core, the body adhesive layer having an inner boundary and an outer boundary, the outer boundary being coextensive with the perimeter of the fabric layer, and the inner boundary circumscribing the exposable portion of the upper surface of the fabric layer, the body adhesive layer abutting the upper surface of the fabric layer and configured to be pressed against and adhered to the user, and
a removable protective layer disposed adjacent to and selectively covering the exposable portion of the upper surface of the fabric layer and removably adhered to the fabric layer by the body adhesive layer;
removing the removable protective layer from the pantiless liner;
positioning the front portion of the pantiless liner such that the inner boundary of the body adhesive layer is positioned adjacent a clitoral hood of user;
positioning the rear portion the pantiless liner such that the body adhesive layer of the pantiless liner is positioned on a portion of a perineum of the user; and
pressing the body adhesive layer against the user, whereby the pantiless liner is adhered to the user.

2. The method of claim 1, wherein the pantiless liner has a length between 4.5 inches and 4.9 inches.

3. The method of claim 1, wherein the moisture-impervious layer includes a material selected from the group consisting of polypropylene, polyethylene, polyurethane, and polyester and is configured to militate against moisture permeating the pantiless liner.

4. The method of claim 1, wherein the fabric layer includes a non-woven fabric configured to absorb discharge of the user.

5. The method of claim 1, wherein the SAP layer includes sodium polyacrylate.

6. The method of claim 1, wherein the transfer adhesive layer includes an acrylic adhesive, and the method further includes a step of laminating the transfer adhesive layer to the portion of the moisture-impervious layer and the core via during a manufacturing process.

7. The method of claim 1, wherein the body adhesive layer comprises a silicone adhesive.

8. The method of claim 1, wherein the body adhesive layer is a bilayer, and the bilayer includes a first sublayer of an acrylic adhesive and a second sublayer of a silicone adhesive, the first sublayer abutting the fabric layer and the second sublayer abutting removable protective layer.

9. The method of claim 1, wherein the fabric layer is embossed with a pattern configured to distribute the moisture or the liquid through and across the fabric layer and the SAP layer.

10. The method of claim 1, wherein the protective layer has a first panel and a second panel, each of the first panel and the second panel disposed lengthwise on the pantiless liner.

11. The method of claim 10, wherein the first panel has a first width and the second panel has a second width, and the first width is greater than the second width, and the first panel has a terminal edge that overlaps the second panel and is configured to assist the user with removal of the protective layer from the pantiless liner.

12. The method of claim 11, wherein the overlap includes a width of 0.2 inches.

13. The method of claim 1, wherein the body adhesive layer has a width between 0.3 inches and 0.5 inches.

14. The method of claim 1, wherein the pantiless liner has a length between 4.2 inches and 5.2 inches, a width between 2 inches and 3 inches, and a thickness between 0.75 millimeters and 1.25 millimeters for adhering the pantiless liner to adjacent the clitoral hood of the user and the portion of the perineum of the user.

15. The method of claim 1, wherein the pantiless liner is one of a plurality of shapes selected from the group consisting of an egg shape, an oval shape, a butterfly shape, a flower shape, lips shape, and a leaf shape.

16. The method of claim 1, wherein the body adhesive layer is configured to be repositioned by the user after initial application without loss of adhesive properties.

17. A pantiless liner for a user, the pantiless liner having a front portion and a rear portion, the pantiless liner comprising:
a moisture-impervious layer being a bottom-most layer of the pantiless liner;
a core including
a super absorbent polymer (SAP) layer including cross-linked polymers configured to absorb liquid, and
a fabric layer disposed adjacent to the SAP layer and including a non-woven material having a lower surface, an upper surface with an exposable portion, and a perimeter;
a transfer adhesive layer disposed between the moisture-impervious layer and the core and coupling a portion of the moisture-impervious layer and the core;
a body adhesive layer disposed on the upper surface and adjacent the perimeter of the fabric layer of the core, the body adhesive layer having an inner boundary and an outer boundary, the outer boundary being coextensive with the perimeter of the fabric layer, and the inner boundary circumscribing the exposable portion of the upper surface of the fabric layer, the body adhesive layer abutting the upper surface of the fabric layer and configured to contact and adhere to the user; and
a removable protective layer disposed adjacent to and selectively covering the exposable portion of the upper surface of the fabric layer and removably adhered to the fabric layer by the body adhesive layer.

18. The pantiless liner of claim 17, wherein the front portion is configured to be positioned adjacent a clitoral hood of user and the rear portion configured to be positioned on a portion of a perineum of the user.

19. A pantiless liner for a user, the pantiless liner having a front portion and a rear portion, the pantiless liner consisting of:
a moisture-impervious layer being a bottom-most layer of the pantiless liner;
a core including
a super absorbent polymer (SAP) layer including cross-linked polymers configured to absorb liquid, and
a fabric layer disposed adjacent to the SAP layer and including a non-woven material having a lower surface, an upper surface with an exposable portion, and a perimeter;
a transfer adhesive layer disposed between the moisture-impervious layer and the core and coupling a portion of the moisture-impervious layer and the core;
a body adhesive layer disposed on the upper surface and adjacent the perimeter of the fabric layer of the core, the body adhesive layer having an inner boundary and an outer boundary, the outer boundary being coextensive with the perimeter of the fabric layer, and the inner boundary circumscribing the exposable portion of the upper surface of the fabric layer, the body adhesive layer abutting the upper surface of the fabric layer and configured to contact and adhere to the user; and
a removable protective layer disposed adjacent to and selectively covering the exposable portion of the upper surface of the fabric layer and removably adhered to the fabric layer by the body adhesive layer.

20. The pantiless liner of claim 19, wherein the front portion is configured to be positioned adjacent a clitoral hood of user and the rear portion configured to be positioned on a portion of a perineum of the user.
